(19) United States
(12) Patent Application Publication
(10) Pub. No.: US 2025/0032105 A1
(43) Pub. Date: Jan. 30, 2025

(54) FLUID MANAGEMENT SYSTEM AND TISSUE TRAP

(71) Applicant: Tamas J. TRUCKAI, Saratoga, CA (US)

(72) Inventor: Tamas J. TRUCKAI, Saratoga, CA (US)

(21) Appl. No.: 18/784,465

(22) Filed: Jul. 25, 2024

Related U.S. Application Data
(60) Provisional application No. 63/515,391, filed on Jul. 25, 2023.

(51) Int. Cl. A61B 17/00

(52) U.S. Cl. CPC A61B 17/00234; A61B 2017/00296; A61B 2217/005

(57) ABSTRACT

Tissue traps for fluid management systems of the type used in medical procedures, for example, in endoscopic procedures.

### CROSS-REFERENCE TO RELATED APPLICATIONS

This application is a non-provisional application of U.S. Provisional application No. 63/515,391 filed Jul. 25, 2023. The entirety of which is incorporated by reference.

### BACKGROUND OF THE INVENTION

The subject matter described herein relates to a fluid management system of the type used, for example, in endoscopic procedures.

Surgical fluid management systems typically deliver a fluid, such as saline, to a targeted working space or body cavity to provide access and visibility to the physician performing a procedure in the space. The fluid usually provides pressure sufficient to “open” the space (i.e., create a working space for the procedure) and additionally will usually flush blood and debris from the space.

Typically, the fluid management system includes a control system for maintaining a preset fluid pressure in a working space.

### BACKGROUND OF THE INVENTION

The present disclosure includes a collection bag or tissue traps for use with a fluid management system configured to collect a liquid outflow containing a tissue debris produced by a tissue resection system.

In one variation, a tissue trap can include a tissue trap body configured for positioning within a fluid collection sac of the fluid management system through an opening of a threaded fitting of the fluid collection sac, the tissue trap body having a bag opening with a trap washer located about the bag opening, such that, when the tissue trap body is positioned within the fluid collection sac, the trap washer is configured for positioning about the threaded fitting to form a seal thereagainst when a locking cap is secured to the threaded fitting to permit liquid outflow to enter the tissue trap and fluid collection sac; wherein a wall material of the tissue trap body comprises a liquid permeable wall portion such that when a liquid and a resected tissue enters the tissue trap body and fluid collection sac, the liquid passes through the liquid permeable wall portion into the fluid collection sac while the tissue debris remains within the tissue trap body, wherein the tissue trap body containing the tissue debris is configured for removal from the fluid collection sac through the threaded fitting while the fluid collection sac remains in place.

Variations of the tissue trap can have a volume sufficient to collect at least 20 grams, at least 30 grams or at least 40 grams of tissue debris.

Additional variations of the tissue traps include the liquid permeable wall portion that comprises a material selected from a group consisting of a cloth, a mesh, a screen or a composite material.

In addition, tissue traps can have an entirety of the wall material that is liquid permeable. Alternatively, the liquid permeable wall portion can be located at a lower end of the tissue trap body such that the liquid drains outflow from the lower end.

The present disclosure also includes methods of removing tissue debris from a liquid outflow of a tissue resection system without removing the liquid outflow from a collection sac of the tissue resection system.

In one variation such a method includes inserting a tissue trap a fluid collection sac of a fluid management system coupled to the tissue resection system through an opening of a threaded fitting of the fluid collection sac; securing a locking cap onto the threaded fitting such that a trap washer located on a bag opening seals between the locking cap and the threaded fitting; capturing liquid outflow from the tissue resection system within the tissue trap and fluid collection sac, such that tissue debris from the liquid outflow remains within the tissue trap and the liquid outflow passes out of a liquid permeable portion of the tissue trap into the fluid collection sac; and removing the tissue trap and the tissue debris from the fluid collection sac through the opening of the threaded fitting.

The methods can include removing the tissue trap and the tissue debris without removing the fluid collection sac from the fluid management system.

### BRIEF DESCRIPTION OF THE FIGURES

FIG. 1 is a perspective view of a fluid management system, including a roll stand with a control unit and inflow and outflow peristaltic pumps, a detachable cassette with a tubing set, and a waste collection sac.

FIG. 2 illustrates an enlarged view of the control unit and peristaltic pumps of FIG. 1 and further shows a tissue trap that can be detachably carried in the waste collection sac.

FIG. 3A is an enlarged view of the tissue trap of FIG. 2, separate from the waste collection sac, a locking cap, and a portion of a wall of the collection sac.

FIG. 3B is another variation of a tissue trap or collection bag where a portion of the bag is liquid permeable and a remainder of the bag is liquid impermeable.

### DETAILED DESCRIPTION

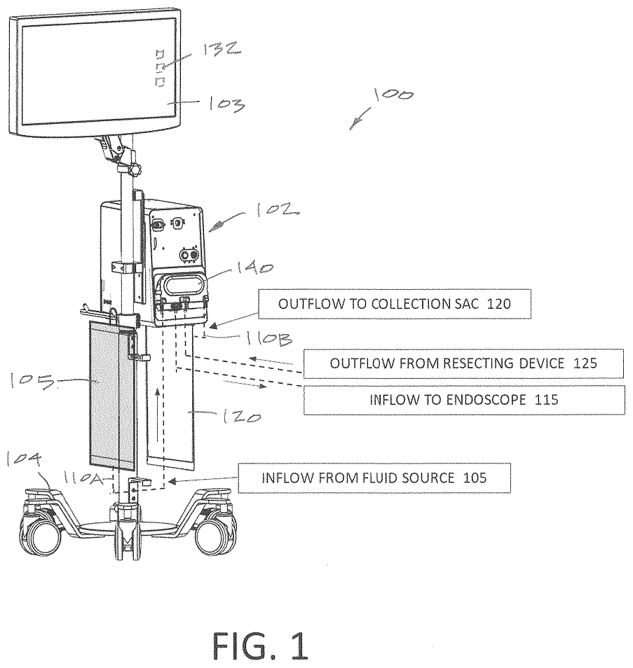

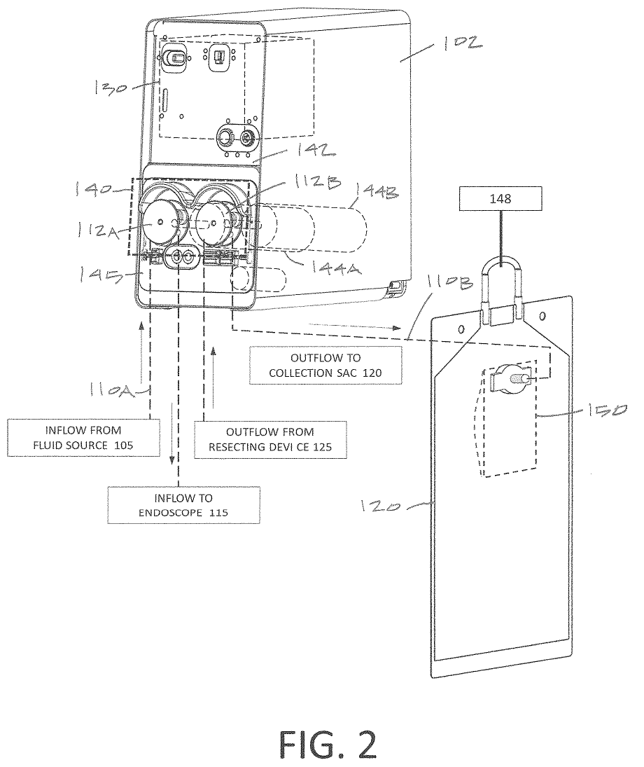

FIG. 1 illustrates a fluid management system 100 of the invention, which includes a control unit 102 and video display 103 mounted on a roll stand 104. The fluid management system 100 is used in endoscopic procedures, which can be a gynecology procedure, a urology surgery, or an arthroscopic surgery, to provide inflows and outflows of a distention fluid to and from a working space, for example, a body cavity. The distention fluid is typically a saline solution and can be delivered through an endoscope or other device to provide and maintain a preset pressure level within the working space. In the system shown in FIG. 1, the fluid is delivered from a fluid source 105, for example, a flexible saline bag connected to inflow tubing 110A that is mated with an inflow peristaltic pump 112A (FIG. 2). The pump 112A is used to provide a fluid flow therefrom through the inflow tubing 110A that is typically coupled to an endoscope 115 with an inflow channel therein.

As also can be understood from FIG. 1, the roll stand 104 further carries a waste collection sac or reservoir 120, which is adapted to receive outflows from a working space through outflow tubing 110B coupled to outflow peristaltic pump 112B (FIGS. 1 and 2) that pumps fluid from a resecting device 125 or other cutting tools. In another variation, the roll stand 104 can carry an independent drape pump (not shown) for removing fluid from a surgical drape that may be used in a surgical procedure. Such a drape pump can be adapted to remove fluid from the drape through additional waste tubing (not shown) to the waste collection sac 120 (FIG. 2). As will be described below, a processor or controller 130 (FIGS. 1-2) in the control unit 102 is configured to sense the weight of fluid in the collection sac 120 from which a fluid deficit can be calculated and which is important for many surgical procedures.

In FIG. 1, one variation of a control unit 102 is coupled to a type of video display 103 that comprises a touch screen. In this variation, various icons 132 on the touch screen display 103 are adapted for selecting a target pressure in the working space, a fluid inflow rate, flush, image capture with the endoscope, light intensity of LEDs carried by the endoscope, and/or other modes of operation. It should be appreciated that a smaller touchscreen can be integrated into the control unit 102 itself.

Alternatively, an endoscope 115, such as a single-use endoscope, is coupled to the control unit 102 and has a control pad with control buttons for adjusting any operating parameter of the system, such as set pressure, inflow rate, image capture, light intensity, flush, and the like. The touch screen or an actuator on the control unit 102 can be used to unlock or lock a cassette 140 (FIG. 1) that carries loops of the inflow and outflow tubing 110A 110B, as will be described further below.

The video display 103 is also configured to receive data from the controller 130 to continuously display the fluid pressure in the working space, the fluid deficit (further described below), the volume of saline remaining in the fluid source or saline bag 105, and the fluid inflow rate. In some variations, the endoscope 115 may be coupled to the controller 130, and LED light intensity may be displayed on the video display 103, as well as other operating parameters of the endoscope.

Now, referring to FIGS. 1 and 2, the control unit 102 has a front surface 142 that carries the inflow and outflow peristaltic roller pumps 110A and 110B with corresponding motors 144A and 144B in the interior of the control unit 102 (see FIG. 2). The peristaltic roller pumps 112A and 112B extend through a slidable base plate 145 in the front surface 142 of the control unit 102. As can be understood from FIGS. 1 and 2, the removable cassette 140 is provided that is adapted for looking in position in the base plate 145 (FIG. 2) to be engaged by the first and second roller pumps 112A and 112B.

In another aspect, the system 100 and controller 130 are capable of determining fluid deficit, which is important in some surgical procedures, such as gynecology. As can be understood from FIGS. 1 and 2, the roll stand 104 and control unit 102 can carry a load sensor 148 (FIG. 2) that provides a signal to the controller 130 of the weight of saline collected in collection sac 120, which is collected from fluid outflows. The fluid deficit monitoring system is calibrated at the start of a procedure by providing the controller 130 with the known volume of saline in the fluid source or saline bag 105. The controller 130 is then configured to calculate the volume of saline inflows to the working space WS and the volume of saline collected in the collection sac 120, from which the fluid deficit can then be calculated. In one variation, the fluid deficit is continuously calculated and shown on the display 103. The system 100 further can include audio and/or visual alarms when the fluid deficit reaches a preselected level, and the system can include a shut-down mechanism when the fluid deficit reaches a preselected level.

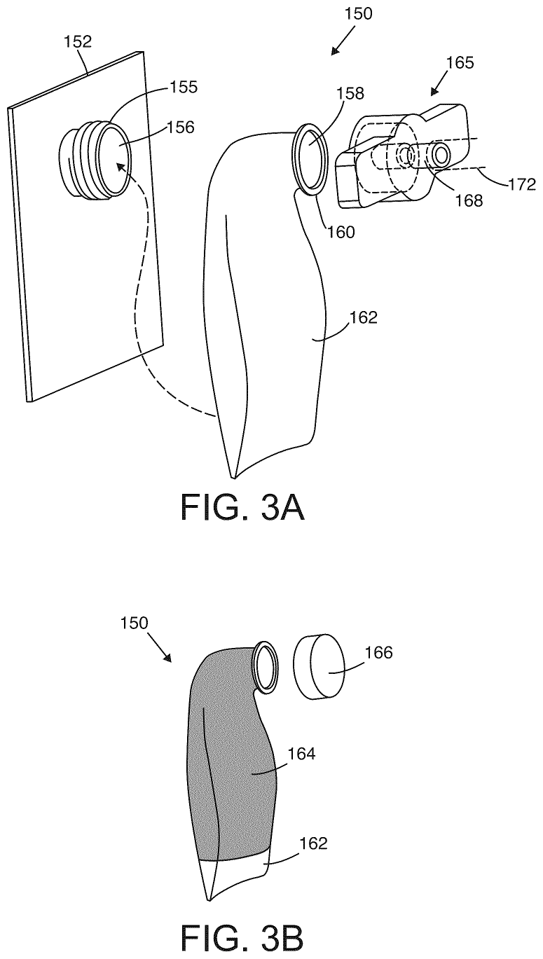

In another aspect of the invention, a tissue trap 150 is shown in FIGS. 2 and 3A that is configured for integration into the collection sac 120. In a typical prior art fluid management system, a plastic tissue trap assembly with a filter is positioned in a portion of the outflow tubing between the outflow pump and the fluid collection reservoir. In the variation of FIGS. 2 and 3A corresponding to the invention, the tissue trap 150 is less costly and has a larger capacity for collecting resected tissue chips than prior art devices. As can be seen in FIGS. 2 and 3A, the tissue trap 150 is configured for positioning inside a wall 152 of thin-wall collection sac 120. Referring to FIG. 3A, a plastic threaded fitting 155 with a large opening 156 is bonded to the wall 152 of the collection sac 120. The wall material 162 of the tissue trap 150 can comprise a liquid permeable material that allows liquid to pass through while retaining tissue and other debris located within the liquid outflow during the procedure. Such liquid permeable materials can include a cloth or mesh material that can be folded or collapsed for insertion through the opening 156 in the threaded fitting 155. The tissue trap 150 has an open end 158 with a trap washer member 160 dimensioned to engage the top of the threaded fitting 155. FIG. 3A further shows a screw-on locking cap 165 that is adapted to screw onto the threaded fitting 155 and seal against trap washer member 160 between the fitting 155 and the locking cap 165. The locking cap 165 has a suitable fitting 168, such as a Luer fitting that receives the distal end 172 of the outflow tubing 110B.

In a variation, the tissue trap 150 is dimensioned to receive at least 20 grams, at least 30 grams, or at least 40 grams of resected tissue chips. As can be understood from FIG. 3A, the tissue trap 150 can easily be removed and replaced quickly during a surgical procedure by simply unscrewing the locking cap 165, inserting a new tissue trap 150 after removing the old trap, and screwing on the locking cap 165.

FIG. 3B shows a variation of a tissue trap 150 where a portion of the tissue trap body comprises a liquid permeable material 162 while a remainder 164 can be liquid impermeable or less liquid permeable than the material 162. In this variation, the liquid permeable material 162 is located at the bottom of the trap 150. However, the liquid impermeable material sections can be positioned in any region of the tissue trap. Moreover, the liquid permeable material 162 can be folded into the impermeable materials 164 during transportation of the trap 150 after it is removed from the system and a cover 166 is secured to the trap.

Although particular embodiments of the present invention have been described above in detail, it will be understood that this description is merely for purposes of illustration, and the above description of the invention is not exhaustive. Specific features of the invention are shown in some drawings and not in others, and this is for convenience only and any feature may be combined with another in accordance with the invention. A number of variations and alternatives will be apparent to one having ordinary skills in the art. Such alternatives and variations are intended to be included within the scope of the claims. Particular features that are presented in dependent claims can be combined and fall within the scope of the invention. The invention also encompasses embodiments as if dependent claims were alternatively written in a multiple dependent claim format with reference to other independent claims.

As for other details of the present invention, materials and manufacturing techniques may be employed within the level of those with skill in the relevant art. The same may hold true with respect to method-based aspects of the invention in terms of additional acts that are commonly or logically employed. In addition, though the invention has been described in reference to several examples, optionally incorporating various features, the invention is not to be limited to that which is described or indicated as contemplated with respect to each variation of the invention.

Various changes may be made to the invention described, and equivalents (whether recited herein or not included for the sake of some brevity) may be substituted without departing from the true spirit and scope of the invention. Also, any optional feature of the inventive variations may be set forth and claimed independently or in combination with any one or more of the features described herein. Accordingly, the invention contemplates combinations of various aspects of the embodiments or combinations of the embodiments themselves, where possible. Reference to a singular item, includes the possibility that there are plural of the same items present. More specifically, as used herein and in the appended claims, the singular forms “a,” “and,” “said,” and “the” include plural references unless the context clearly dictates otherwise.

It is important to note that, where possible, aspects of the various described embodiments or the embodiments themselves can be combined. Where such combinations are intended to be within the scope of this disclosure.

We claim:

1. A tissue trap for use with a fluid management system configured to collect a liquid outflow containing a tissue debris produced by a tissue resection system, the tissue trap comprising:
a tissue trap body configured for positioning within a fluid collection sac of the fluid management system through an opening of a threaded fitting of the fluid collection sac, the tissue trap body having a bag opening with a trap washer located about the bag opening, such that, when the tissue trap body is positioned within the fluid collection sac, the trap washer is configured for positioning about the threaded fitting to form a seal thereagainst when a locking cap is secured to the threaded fitting to permit liquid outflow to enter the tissue trap and fluid collection sac;
wherein a wall material of the tissue trap body comprises a liquid permeable wall portion such that when a liquid and a resected tissue enters the tissue trap body and fluid collection sac, the liquid passes through the liquid permeable wall portion into the fluid collection sac while the tissue debris remains within the tissue trap body, wherein the tissue trap body containing the tissue debris is configured for removal from the fluid collection sac through the threaded fitting while the fluid collection sac remains in place.

2. The tissue trap of claim 1, wherein a volume of the tissue trap body is configured to collect at least 20 grams, at least 30 grams or at least 40 grams of tissue debris.

3. The tissue trap of claim 1, wherein the liquid permeable wall portion comprises a material selected from a group consisting of a cloth, a mesh, a screen or a composite material.

4. The tissue trap of claim 1, wherein an entirety of the wall material of the tissue trap body is liquid permeable.

5. The tissue trap of claim 1, wherein the liquid permeable wall portion is located at a lower end of the tissue trap body such that the liquid drains outflow from the lower end.

6. A method of removing a tissue debris from a liquid outflow of a tissue resection system, the method comprising:
inserting a tissue trap a fluid collection sac of a fluid management system coupled to the tissue resection system through an opening of a threaded fitting of the fluid collection sac;
securing a locking cap onto the threaded fitting such that a trap washer located on a bag opening seals between the locking cap and the threaded fitting;
capturing liquid outflow from the tissue resection system within the tissue trap and fluid collection sac, such that tissue debris from the liquid outflow remains within the tissue trap and the liquid outflow passes out of a liquid permeable portion of the tissue trap into the fluid collection sac; and
removing the tissue trap and the tissue debris from the fluid collection sac through the opening of the threaded fitting.

7. The method of claim 6, wherein removing the tissue trap and the tissue debris occurs without removing the fluid collection sac from the fluid management system.

8. The method of claim 6, wherein a volume of the tissue trap is configured to collect at least 20 grams, at least 30 grams or at least 40 grams of tissue debris.

9. The method of claim 6, wherein the liquid permeable portion of the tissue trap comprises a material selected from a group consisting of a cloth, a mesh, a screen or a composite material.

10. The method of claim 6, wherein an entirety of the tissue trap is liquid permeable.

11. The method of claim 6, wherein the liquid permeable portion is located at a lower end of the tissue trap such that the liquid outflow drains from the lower end.
